(19) United States
(12) Patent Application Publication
(10) Pub. No.: US 2022/0170756 A1
(43) Pub. Date: Jun. 2, 2022

(54) SYSTEM AND METHOD FOR GENERATING AND UTILIZING MAP DATA INDICATIVE OF HEALTH INFECTION CONDITIONS

(71) Applicant: HERE Global B.V., Eindhoven (NL)

(72) Inventors: Haquemobassir IMTIYAZ SHAIKH, Maharashtra (IN); Richa Rupani, Maharashtra (IN)

(21) Appl. No.: 17/110,019

(22) Filed: Dec. 2, 2020

(51) Int. Cl. G01C 21/34; G01N 33/497; G16H 50/80; G16H 10/60; H04W 4/44; A61L 2/04

(52) U.S. Cl. CPC G01C 21/3461; G01N 33/497; A61L 2/04; G16H 10/60; H04W 4/44; G16H 50/80

(57) ABSTRACT

A system may be provided for generating map data. The system may include a memory configured to store computer executable instructions and a processor configured to execute the computer executable instructions to obtain sensor data. The processor may be further configured execute the computer executable instructions to determine health infection conditions in the geographic region based on the sensor data. The processor may be further configured to generate the map data for the geographic region based on the determined health infections condition in the geographic region.

### TECHNOLOGICAL FIELD

The present disclosure generally relates to navigation systems, and more particularly relates to generating map data that indicates presence of health infection conditions for a geographic region.

### BACKGROUND

Navigation service providers are increasingly providing enhanced services related to navigation, route planning, route optimization and special conditions considerations during navigation. In the current situation, prevalence of diseases and pandemics has even necessitated service providers to think beyond convention. One special condition could be presence of pathogens in air, which is problematic for several reasons. Such pathogens can be related to dangerous diseases and may be transmitted into a passenger compartment of a vehicle when a user, such as the passenger, travels in the vehicle. Further, the pathogens themselves may be blown into the passenger compartment, leading to passenger infection (such as, pneumonia, bronchitis) or other ailments that can be lethal to passengers (such as, covid virus infection). The pathogens, such as the covid virus, can stay in the air for 3 to 4 hours, covered in heavy invisible water droplets. The problem is that such a virus cannot be sensed and seen to avoid, resulting in infections. In certain scenarios, when the vehicle transverses on a road where the air is infected from the pathogens, the passengers may contract an infection that enters the passenger compartment of the vehicle through windows or the air conditioning unit of the vehicle.

Accordingly, there is a need to address the foresaid problems using an advanced vehicle support system.

### BRIEF SUMMARY

In accordance with various embodiments of the present disclosure, an advanced inbuilt vehicle system is provided, which supports in detecting and reacting to presence of pathogens in an environment. Further, the various embodiments provide navigation support services that indicate presence of health infection conditions in the navigation information, using a mapping platform related to the navigation services. The mapping platform comprises map data, which is a map layer or a record of data, that is specifically related to health related information. In some embodiments, this health related information relates to information related to presence of pathogens in air for a geographic region identified in the mapping platform. In several embodiments, the geographic is further construed as comprised of road segments, lanes, and other road geometries, and the mapping platform includes the map layer which stored health related information for each of these road segments, lanes and road geometries.

A system is provided herein that focuses on generating map layer indicative of health infection conditions in the geographic region. The system may include at least one non-transitory memory configured to store computer executable instructions, and at least one processor (hereinafter referred as processor) configured to execute the computer executable instructions to obtain sensor data for the geographic region. In accordance with an embodiment, the processor may be configured to execute the computer executable instructions to determine a health infection conditions in the geographic region based on the sensor data. The processor may be configured to execute the computer executable instructions to generate the map data for the geographic region based on the determined health infection condition in the geographic region. In accordance with an embodiment, the sensor data comprises location data, data associated with presence of pathogens in air for the geographic region, or a combination thereof. In accordance with an embodiment, the pathogens are associated with coronavirus family of viruses. In accordance with an embodiment, the system comprises one or more specialized air sensors to obtain the data associated with the presence of the pathogens in the air for the geographic region.

In accordance with an embodiment, the data associated with the presence of the pathogens in air for the geographic region comprises at least one of: news related to prevalence of a disease in the geographic region, wherein the disease is Covid-19; medical records associated with health conditions of population in the geographic region; government records associated with presence of the health infection in the geographic region; and social network records of the population in the geographic region associated with the health infection.

According to some example embodiments, the processor may be further configured to execute the computer executable instructions to map match location data associated with the geographic region to a specific location of map data for the geographic region. According to some example embodiments, the processor may be further configured to execute the computer executable instructions to generate a health data record for the geographic region, based on the determined health infection conditions in the geographic region.

According to some example embodiments, the processor may be further configured to execute the computer executable instructions to generate the health data record for at least one of: a map tile area, a lane, a road segment, a road geometry, a shape location data record, a street, a locality, a point of interest (POI) record, a route of travel and a destination location.

According to some example embodiments, the processor may be further configured to execute the computer executable instructions to transmit a warning message to at least one vehicle for indicating presence of the health infection condition on a road, based on the map data.

According to some example embodiments, the processor may be further configured to execute the computer executable instructions to recommend driving strategies to the at least one vehicle, based on the map data.

According to some example embodiments, the processor may be further configured to execute the computer executable instructions to transmit a report of the generated map data to a backend server.

According to some example embodiments, the processor may be further configured to execute the computer executable instructions to control navigation based services for at least one vehicle, based on the generated map data.

According to some example embodiments, the processor may be further configured to execute the computer executable instructions to re-route the at least one vehicle in the geographic region, based on the navigation based services.

According to some example embodiments, the processor may be further configured to execute the computer executable instructions to control operations of the at least one vehicle for selecting a vehicle functionality, based on the navigation based services.

In accordance with an embodiment, the vehicle functionality comprises switching off an air conditioning unit of the at least one vehicle, based on the navigation based services, wherein the switching off the air conditioning unit prevents infected air from entering into a passenger compartment of the at least one vehicle. In accordance with an embodiment, the vehicle functionality further comprises switching on a heat chamber of the at least one vehicle, based on the navigation based services, wherein the heat chamber is configured to destroy the pathogens entering the at least one vehicle from infected air at a predefined temperature and predefined pressure. In accordance with an embodiment, the sensor data further comprises size data of water droplets and volume data of the water droplets, wherein a water droplet size below a threshold range is indicative of the health infection conditions.

Embodiments of the present disclosure may provide a computer programmable product including at least one non-transitory computer-readable storage medium having computer-executable program code stored therein. The computer programmable product comprising a non-transitory computer readable medium having stored thereon computer executable instructions, which when executed by a computer, cause the computer to carry out operations, for providing navigation services in a geographic region, the operations comprising obtaining, by one or more processors, receiving, from at least one vehicle, input data associated with a route of travel in the geographic region; determining, for the input data associated with the route of travel, map data indicative of health infection conditions in the geographic region; and providing the navigation services to the at least one vehicle, based on the map data.

Embodiments of the present disclosure may provide a method for operating at least one vehicle, the method comprising obtaining, information associated with a route of travel, for the at least one vehicle; receiving, from a map database, a route response comprising route information indicative of presence of health infection conditions on the route of travel of the at least one vehicle; and operating the at least one vehicle by controlling a vehicle functionality of the at least one vehicle, based on the received route response.

In accordance with an embodiment, a system is provided wherein the system may obtain sensor data indicative of infected air conditions for a geographic region, social information associated with presence of a health infection in the geographic region, or a combination thereof. The system may be configured to generate the map data for the geographic region based on the sensor data and the social information.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

Having thus described example embodiments of the disclosure in general terms, reference will now be made to the accompanying drawings, which are not necessarily drawn to scale, and wherein:

FIG. 1 is a block diagram that illustrates a network environment of a mapping platform implemented for generating map data indicative of health infection conditions, in accordance with an example embodiment;

FIG. 2 illustrates a block diagram of the mapping platform, exemplarily illustrated in FIG. 1, that may be used for generating map data indicative of health infection conditions, in accordance with an example embodiment;

FIG. 3 illustrates a block diagram of a map database used to store data, such as sensor data for generation of map data, in accordance with an embodiment;

FIG. 4 is a schematic diagram that exemplarily illustrates a scenario for updating health data records of a map database, in accordance with an example embodiment;

FIG. 5 is a schematic diagram that exemplarily illustrates a scenario for a vehicle that uses a navigation based service indicative of health infection conditions in a geographic region, in accordance with an embodiment;

FIG. 6 illustrates a flowchart for implementation of an exemplary method to generate map data indicative of health infection conditions in a geographic region, in accordance with an example embodiment;

FIG. 7 illustrates a flowchart for implementation of an exemplary method for providing navigation services in a geographic region, in accordance with an example embodiment; and

FIG. 8 illustrates a flowchart for implementation of an exemplary method for operating a vehicle, in accordance with an example embodiment.

### DETAILED DESCRIPTION

In the following description, for purposes of explanation, numerous specific details are set forth in order to provide a thorough understanding of the present disclosure. It will be apparent, however, to one skilled in the art that the present disclosure may be practiced without these specific details. In other instances, systems and methods are shown in block diagram form only in order to avoid obscuring the present disclosure.

Some embodiments of the present disclosure will now be described more fully hereinafter with reference to the accompanying drawings, in which some, but not all, embodiments of the disclosure are shown. Indeed, various embodiments of the disclosure may be embodied in many different forms and should not be construed as limited to the embodiments set forth herein; rather, these embodiments are provided so that this disclosure will satisfy applicable legal requirements. Like reference numerals refer to like elements throughout. Also, reference in this specification to “one embodiment” or “an embodiment” means that a particular feature, structure, or characteristic described in connection with the embodiment is included in at least one embodiment of the present disclosure. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity, but rather denote the presence of at least one of the referenced item. Moreover, various features are described which may be exhibited by some embodiments and not by others. Similarly, various requirements are described which may be requirements for some embodiments but not for other embodiments. As used herein, the terms “data,” “content,” “information,” and similar terms may be used interchangeably to refer to data capable of being displayed, transmitted, received and/or stored in accordance with embodiments of the present disclosure. Thus, use of any such terms should not be taken to limit the spirit and scope of embodiments of the present disclosure.

As defined herein, a “computer-readable storage medium,” which refers to a non-transitory physical storage medium (for example, volatile or non-volatile memory device), may be differentiated from a “computer-readable transmission medium,” which refers to an electromagnetic signal.

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present disclosure. Further, it is to be understood that the phraseology and terminology employed herein are for the purpose of the description and should not be regarded as limiting. Any heading utilized within this description is for convenience only and has no legal or limiting effect.

### Definitions

The term “road” may refer to a way leading a traveler from one location to another. The road may have a single lane or multiple lanes.

The term “lane” may refer to a part of a road that is designated for travel of vehicles or pedestrians as per some condition.

The term “link” may refer to any connecting pathway including, but not limited, to a roadway, a highway, a freeway, an expressway, a lane, a street path, a road, an alley, a controlled access roadway, a free access roadway and the like.

The term “route” may refer to a path from a source location to a destination location on any link.

The term “autonomous vehicle” may be used to refer to any vehicle having autonomous driving capabilities at least in some conditions. An autonomous vehicle, as used throughout this disclosure, may also be known as a driverless car, robot car, self-driving car or autonomous car. For example, the vehicle may have zero passengers or passengers that do not manually drive the vehicle, but the vehicle drives and maneuvers automatically. There can also be semi-autonomous vehicles and manual vehicles.

### End of Definitions

A system is provided herein that focuses on generating map data, including a map layer, indicative of health infection conditions in the geographic region. The system disclosed herein provides accurate and precise map data for providing high quality navigation assistance, especially when the route has one or more road segments with infected air conditions, in near real time. The infected air conditions may result in health infection conditions in persons contracting infection by taking such routes. The system disclosed herein facilitates safety health issues and alert drivers to health infection conditions in a timely and targeted way in advance. The system disclosed herein may be configured to generate a map layer for map data stored in a map database that indicates health infection conditions in the geographic region.

The system disclosed herein may be configured to update the map data based on the generated map layer. The system disclosed herein may further provide a notification message associated with the health infection conditions on the road. For example, the notification message may inform a vehicle or user equipment with up-to-date map data for the region that indicates presence of health infection conditions. Alternatively, the available up-to-date data may be pushed as an update to the vehicle or the user equipment.

Further embodiments provide a method for operating a vehicle in accordance with the functionalities provided by the system disclosed herein. The method disclosed herein may be configured to operate at least one vehicle. In accordance with an embodiment, the vehicle may receive, from a map database, a route response comprising route information indicative of presence of health infection conditions on the route of travel of the at least one vehicle. Vehicle functionality of the vehicle may be controlled based on the route information indicative of the presence of health infection conditions on the route of travel. The method disclosed herein facilitates alerting drivers to health infection conditions, such that the vehicle functionality may be controlled in a timely and targeted way in advance.

Further embodiments provide a computer program product including at least one non-transitory computer-readable storage medium having computer-executable program code stored therein, which when executed by a computer, cause the computer to carry out operations, for providing navigation services in a geographic region. The computer program product disclosed herein may be configured to carry out operations to provide navigation services in a geographic region indicative of presence of health infection conditions on a route of travel. These and other technical improvements of the present disclosure will become evident from the description provided herein.

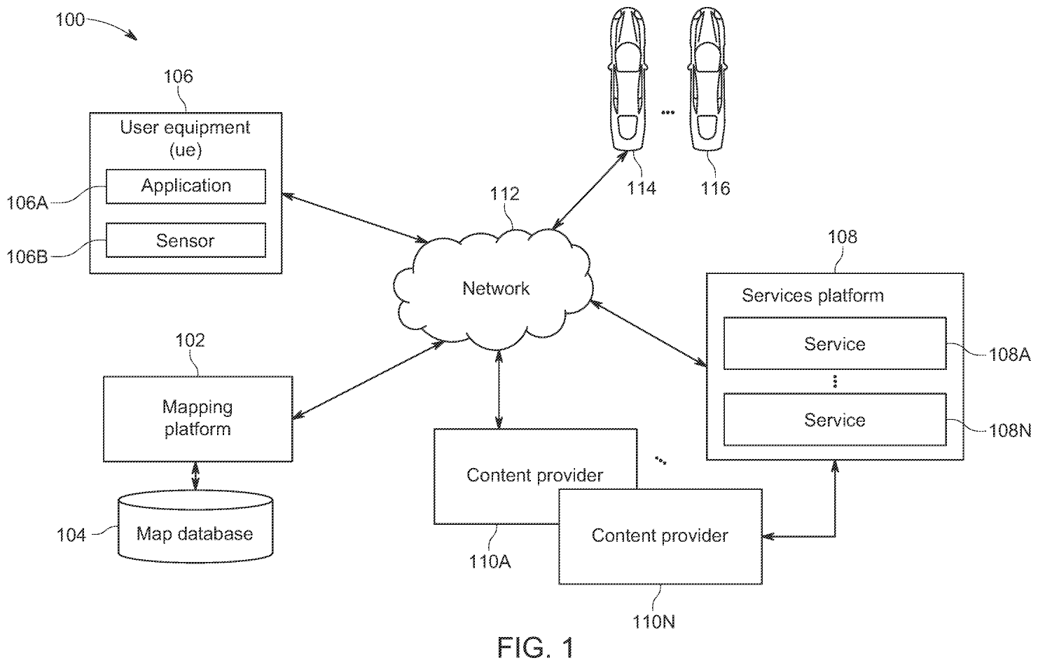

FIG. 1 is a block diagram that illustrates a network environment of a system implemented for generating map data associated with a road in a geographic region, in accordance with an example embodiment.

There is shown a network environment 100 that may include a mapping platform 102, a map database 104, user equipment (UE) 106, a services platform 108 with a plurality of services 108A . . . 108N, a plurality of content providers 110A . . . 110N, and a network 112. There is further shown one or more vehicles, such as a first vehicle 114 and a second vehicle 116 on a road in a region. The first vehicle 114 and the second vehicle 116 may be each be configured as at least one of the autonomous vehicles, the semi-autonomous vehicle or the manual vehicle described earlier. When the one or more vehicles are configured to be autonomous vehicles, some or all of the functionalities related to vehicle navigation may be performed by the vehicle itself, such as through an Advanced Driving Assistance System (ADAS), which may identify various maneuvers for vehicle control, operation and navigation. The UE 106 may be configured as the ADAS to provide autonomous vehicle capabilities to any of the vehicles 114 and 116, in which the UE 106 is installed.

The UE 106 may include an application 106A, a sensor 106B and a user interface (not shown in the FIG. 1). The mapping platform 102 may be communicatively coupled to the UE 106, via the network 112. As per specific requirements, the mapping platform 102 may also be communicatively coupled to other components not shown on FIG. 1 via the network 112.

In some example embodiments, the mapping platform 102 may be implemented in a cloud computing environment. In some other example embodiments, the mapping platform 102 may be implemented in the first vehicle 114. In accordance with an embodiment, the mapping platform 102 may communicate directly with the map database 104. In accordance with another embodiment, the map database 104 may be a part of the mapping platform 102. All the components in the network environment 100 may be coupled directly or indirectly to the network 112. The components described in the network environment 100 may be further broken down into more than one component and/or combined together in any suitable arrangement. Further, one or more components may be rearranged, changed, added, and/or removed.

The mapping platform 102 may comprise suitable logic, circuitry, and interfaces that may be configured to generate map data that indicates health infection conditions for the geographic region. The mapping platform 102 may be configured to obtain sensor data for the geographic region. In accordance with an embodiment, the sensor 106B may be configured to collect sensor data in the geographic region. In accordance with an embodiment, the sensor 106B may be deployed in the UE associated with the first vehicle 114.

In addition or as an alternate to collecting sensor data from the sensors 106B, the mapping platform 102 may also use other sensor data collected in the geographic region using any other equivalent means (such as, from third party vendors). For example, the sensor data of the geographic region can be determined from location trace or probe data collected or reported from probe vehicles as they enter in or leave the geographic region. In accordance with an embodiment, the mapping platform 102 may be further configured to determine health infection conditions in the geographic region based on the sensor data.

The mapping platform 102 may be further configured to generate the map data for the geographic region, based on the sensor data and the map data. Additionally, or alternatively, the mapping platform 102 may be configured to receive geo-coordinates of the geographic region from map data stored in the map database 104.

The generated map data may be indicative of one or more map attributes associated with the road in the geographic region. The mapping platform 102 may be configured to update the map data, in the map database 104, associated with the generated map data. The mapping platform 102 may include techniques related to, but not limited to, geocoding, routing (multimodal, intermodal, and unimodal), convolutional algorithms, clustering algorithms, machine learning in location based solutions, natural language processing algorithms, and artificial intelligence algorithms. Data for different modules of the mapping platform 102 may be collected using a plurality of technologies including, but not limited to drones, sensors, connected cars, cameras, probes, and chipsets. In some embodiments, the mapping platform 102 may be embodied as a chip or chip set. In other words, the mapping platform 102 may comprise one or more physical packages (such as, chips) that includes materials, components and/or wires on a structural assembly (such as, a baseboard).

The map database 104 may comprise suitable logic, circuitry, and interfaces that may be configured to store the map data and the sensor data which may be collected, for example, from one or more vehicles traveling along a road network or within a venue. The sensor data may be gathered and fused to infer an accurate map of an environment in which probes or fleeting cars are moving. In accordance with an embodiment, such sensor data may be updated in real time or near real time such as on an hourly basis, to provide accurate and up to date sensor data. The sensor data may be collected from any sensor that may inform the mapping platform 102 associated with the map database 104 of features within an environment that are appropriate for navigation related services. In accordance with an embodiment, the sensor data may be collected from any sensor that may inform the map database 104 of features within an environment that are appropriate for mapping. For example, motion sensors, inertia sensors, image capture sensors, proximity sensors, LIDAR (light detection and ranging) sensors, and ultrasonic sensors may be used to collect the probe data. The gathering of large quantities of crowd-sourced data may facilitate the accurate modeling and mapping of an environment, whether it is a road link or an interior of a multi-level parking structure.

In accordance with an embodiment, the sensor data (such as, floating car data) may be collected from consumer vehicles traveling on the road throughout a geographic region (or a region). In accordance with an embodiment, a map developer may employ field personnel to travel by a vehicle along roads throughout the region to observe features and/or record information. The map developers may crowdsource geographic map data (or the map data) and vehicle sensor data (or the sensor data) to generate, substantiate, or update the map data in the map database 104. In accordance with an embodiment, the sensor data may comprise location data, data associated with presence of pathogens in air for the geographic region and a combination thereof. In accordance with an embodiment, the pathogens may be associated with coronavirus family of viruses. In accordance with an embodiment, the data associated with the presence of the pathogens in air for the geographic region comprises at least one of: news related to prevalence of a disease in the geographic region. The disease may be Covid-19, medical records associated with health conditions of population in the geographic region, government records associated with presence of the health infection in the geographic region, and social network records of the population in the geographic region associated with the health infection. In accordance with an embodiment, the sensor data may comprise size data of water droplets and volume data of the water droplets. A water droplet size below a threshold range may be indicative of the health infection conditions.

The sensor data may be used by the mapping platform 102 to generate map data that indicates health infection conditions associated with movement of one or more vehicles along the road in the geographic region. The sensor data may be observed within the given time period and projected to determination of health air conditions for that given time period. In accordance with an embodiment, the sensor data may further include, but not limited to, presence of water droplets of certain size in environment, real time speed, incident data on the road, road closure and construction data.

In accordance with an embodiment, the sensor data from the map database 104 may also be supplemented with additional ground truth data acquired from roadside sensors. Advanced planning and coordination for huge volume of water droplets in air that results in the health infection conditions may allow agencies to develop and deploy optimal operational strategies, traffic control plans, protocols, procedures, and technologies needed to reroute traffic and share real time or near real time information with other stakeholders on the day of severe health infection conditions. Such capabilities may allow agencies to proactively manage and control traffic to accommodate or avoid route towards road of infected air. The route may be optimized by controlling vehicle functionality on the road with the infected air. The vehicle functionality may include switching off an air conditioning system of the vehicle (such as the first vehicle 114 or the second vehicle 116) while passing on the road with infected air so that the infected air does not pass into passenger compartment of the vehicle (such as, the first vehicle 114) via the air conditioning system. Further, in accordance with an embodiment, the vehicle functionality of the vehicle (such as, the first vehicle 114) may be controlled by the mapping platform 102 by switching on a heat chamber of the vehicle (such as, the first vehicle 114). The heat chamber of the first vehicle 114 may be configured to destroy the pathogens, such as corona virus present in the infected air, by subjecting the pathogens to very high temperature and high pressure as compared to the temperature and pressure of the outside environment. In accordance with an embodiment, the map database 104 may be configured to store sensor data for size and volume of water droplets present in the environment for heavy rains, snow, and fog to differentiate such water droplets from the ones that are carrying the virus.

In accordance with an embodiment, the map data in the map database 104 may also include cartographic data, routing data, and maneuvering data. The map data may also include, but not limited to, locations of intersections, diversions to be caused due to the infected air conditions, suggested routes to avoid infected air conditions on the road. In accordance with an embodiment, the map database 104 may be configured to receive the sensor data related to the infected air conditions in the region for a road network from external systems, such as, one or more of background batch data services, streaming data services and third party service providers, via the network 112.

In some embodiments, the map database 104 may be a part of the mapping platform 102. The map database 104 may be a master map database stored in a format that facilitates updating, maintenance, and development. For example, the master map database or data in the master map database may be in an Oracle spatial format or other spatial format, such as, for development or production purposes. The Oracle spatial format or development/production database may be compiled into a delivery format, such as a geographic data files (GDF) format. The data in the production and/or delivery formats may be compiled or further compiled to form geographic database products or databases, which may be used in end user navigation devices or systems.

In addition, the map database 104 may include the sensor data for the events (such as, but not limited to, traffic incidents, construction activities, scheduled events, and unscheduled events) associated with Point of Interest (POI) data records or other records of the map database 104 associated with the mapping platform 102.

For example, geographic data may be compiled (such as into a platform specification format (PSF)) to organize and/or configure the data for performing navigation-related functions and/or services, such as route calculation, route guidance, map display, speed calculation, distance and travel time functions, and other functions, by a navigation device, such as the UE 106. The navigation-related functions may correspond to vehicle navigation, pedestrian navigation, navigation to a favored parking spot or other types of navigation. While example embodiments described herein generally relate to vehicular travel and parking along roads, example embodiments may be implemented for bicycle travel along bike paths and bike rack/parking availability, boat travel along maritime navigational routes including dock or boat slip availability, etc. The compilation to produce the end user databases may be performed by a party or entity separate from the map developer. For example, a customer of the map developer, such as a navigation device developer or other end user device developer, may perform compilation on a received map database 104 in a delivery format to produce one or more compiled navigation databases.

In some embodiments, the map database 104 may be a master geographic database configured on the side of the mapping platform 102. In accordance with an embodiment, a client-side map database may represent a compiled navigation database that may be used in or with end user devices (e.g., the UE 106) to provide navigation service based on the infected air condition, the traffic conditions, speed adjustment, and/or map-related functions to navigate through the road in the region.

Optionally, the map database 104 may contain lane segment and node data records or other data that may represent the road segment in the region, pedestrian lane or areas in addition to or instead of the vehicle road record data. The road segments and nodes may be associated with attributes, such as geographic coordinates, street names, address ranges, speed limits, turn restrictions at intersections, and other navigation related attributes, as well as POIs, such as fueling stations, hotels, restaurants, museums, stadiums, offices, auto repair shops, buildings, stores, and parks. The map database 104 may additionally include data about places, such as cities, towns, or other communities, and other geographic features such as, but not limited to, bodies of water, and mountain ranges.

The UE 106 may comprise suitable logic, circuitry, and interfaces that may be configured to provide navigation assistance to vehicles, such as, the first vehicle 114 among other services. In accordance with an embodiment, the UE 106 may be configured to provide navigation and map functions (such as, guidance and map display) along with the infected air conditions of a route for an end user (not shown in the FIG. 1). The health infection conditions may indicate presence of pathogens in the environment associated with the road. In accordance with an embodiment, the UE 106 may be configured to transmit health infection risk warning message to the first vehicle 114 and the second vehicle, based on the generated map data. In accordance with an embodiment, the UE 106 may be configured to notify the first vehicle 114 with driving strategies to drive on a desired location. The UE 106 may be a part of the first vehicle 114. The UE 106 may be installed in the first vehicle 114. In accordance with an embodiment, the UE 106 may be the vehicle itself.

The UE 106 may include the application 106A with the user interface. In accordance with an embodiment, the UE 106 may be an in-vehicle navigation system, such as, an infotainment system, a personal navigation device (PND), a portable navigation device, a cellular telephone, a smart phone, a personal digital assistant (PDA), a watch, a camera, a computer, a workstation, and other device that may perform navigation-related functions (such as digital routing and map display). Examples of the UE 106 may include, but is not limited to, a mobile computing device (such as a laptop computer, tablet computer, mobile phone and smart phone), navigation unit, personal data assistant, watch, and camera. Additionally, or alternatively, the UE 106 may be a fixed computing device, such as a personal computer, computer workstation, kiosk, office terminal computer or a system.

In accordance with an embodiment, the UE 106 may be an in-vehicle navigation system for navigation and map functions (such as, guidance and map display). The UE 106 may include the application 106A with the user interface to access one or more map and navigation related functions that may include infected air conditions notification rendered by the mapping platform 102. In other words, the UE 106 may include the application 106A with the user interface. The user interface may be configured to enable the end user associated with the UE 106 to access the mapping platform 102. In accordance with an embodiment, the UE 106 may be accessible to the mapping platform 102 via the network 112. Although a single UE 106 is shown in the example environment 100 of FIG. 1, it may however be contemplated that more than one user equipment may also be possible within the scope of this disclosure and therefore, the mapping platform 102 may be communicatively coupled to as many user equipment as may be required for a specific implementation. In some example embodiments, the UE 106 may serve the dual purpose of a data gatherer and a beneficiary device.

In some example embodiments, the UE 106 may comprise acoustic sensors such as a microphone array, position sensors such as a GPS sensor, orientation sensors such as gyroscope, motion sensors such as accelerometer, a display enabled user interface such as a touch screen display, and other components as may be required for specific functionalities of the UE 106. In some embodiments, the mapping platform 102 may be implemented in the UE 106. Therefore, a local copy of the map data is stored in cache memory of the UE 106.

The network 112 may comprise suitable logic, circuitry, and interfaces that may be configured to provide a plurality of network ports and a plurality of communication channels for transmission and reception of data, such as data from the map database 104. Each network port may correspond to a virtual address (or a physical machine address) for transmission and reception of communication data. For example, the virtual address may be an Internet Protocol Version 4 (IPv4) (or an IPv6 address) and the physical address may be a Media Access Control (MAC) address. The network 112 may include a medium through which the mapping platform 102, and/or the other components may communicate with each other. The network 112 may be associated with an application layer for implementation of communication protocols based on one or more communication requests from at least one of the one or more communication devices. The communication data may be transmitted or received, via the communication protocols. Examples of such wired and wireless communication protocols may include, but are not limited to, Transmission Control Protocol and Internet Protocol (TCP/IP), User Datagram Protocol (UDP), Hypertext Transfer Protocol (HTTP), File Transfer Protocol (FTP), ZigBee, EDGE, infrared (IR), IEEE 802.11, 802.16, cellular communication protocols, and/or Bluetooth (BT) communication protocols.

Examples of the network 112 may include, but is not limited to a wireless channel, a wired channel, a combination of wireless and wired channel thereof. The wireless or wired channel may be associated with a network standard which may be defined by one of a Local Area Network (LAN), a Personal Area Network (PAN), a Wireless Local Area Network (WLAN), a Wireless Sensor Network (WSN), Wireless Area Network (WAN), 5G Network, Wireless Wide Area Network (WWAN), a Long Term Evolution (LTE) network, a plain old telephone service (POTS), and a Metropolitan Area Network (MAN). Additionally, the wired channel may be selected on the basis of bandwidth criteria. For example, an optical fiber channel may be used for a high bandwidth communication. Further, a coaxial cable-based or Ethernet-based communication channel may be used for moderate bandwidth communication.

In operation, the mapping platform 102 may be configured to obtain the sensor data associated with the road in the geographic region. In accordance with an embodiment, the region may be determined by the mapping platform 102 in response to a request from the end user. In accordance with another embodiment, the determination of the region may also be initiated autonomously by the mapping platform 102. The region may be of any size or shape, such as, a rectangle, a circle, a trapezoidal, a square, a triangle, and a parallelogram. In accordance with an embodiment, the sensor data may be classified as current sensor data and historical probe data.

The one or more vehicles (such as the first vehicle 114 and the second vehicle 116) may include, but not limited to, a bus, a taxi, and a vehicle. In accordance with an embodiment, any machine learning technique (such as, deep machine learning technique) may be used to identify the one or more vehicles on the road in the geographic region.

The mapping platform 102 may be configured to determine health infection conditions in the geographic region based on the sensor data. The mapping platform 102 may be configured to generate the map data for the geographic region, based on the sensor data and the map data. The mapping platform 102 may be configured to generate the map data based on the determine health infection conditions in the geographic region.

Further, in accordance with an embodiment, the mapping platform 102 may be configured to update the map data in the map database 104, based on the generated map data. Therefore, the mapping platform 102 may be configured to transmit a warning message for the health infection conditions to the first vehicle 114, based on the updated map data. Further, the mapping platform 102 may be configured to recommend driving strategies to the one or more end user vehicles, based on the updated map data. Such transmission of the warning messages and the driving strategies to drivers of vehicles in a timely manner, may allow drivers to adjust their travel plans to increase safety for health issues and convenience.

Further, a user of a vehicle (such as the first vehicle 114) can make an appropriate decision to drive safely without risking to health infection conditions by changing vehicle functionality, avoid infected air conditions on the road and identify the best route, choose an optimized route based on the generated map data indicative of the health infection conditions. In an embodiment, the driver of the first vehicle 114 may change maneuver, based on the generated map data to drive safely and thereby creating a safer city.

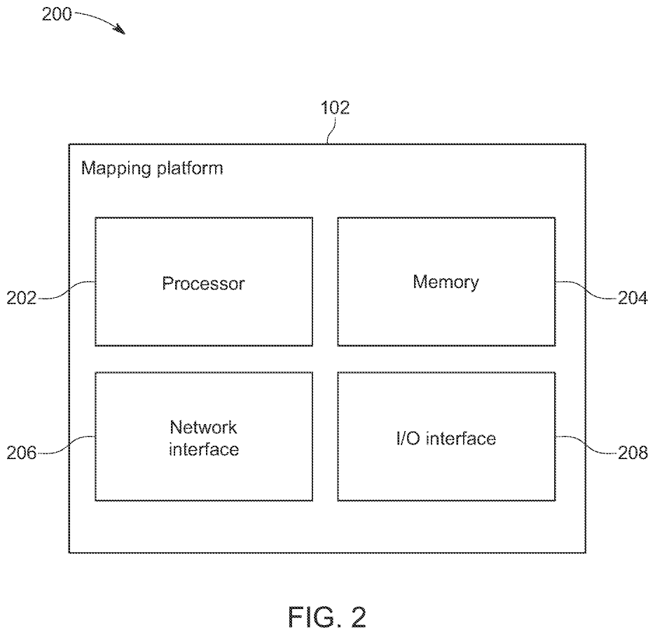

FIG. 2 illustrates a block diagram 200 of the mapping platform, exemplarily illustrated in FIG. 1 that may be used for generating map data for a road in a geographic region, in accordance with an example embodiment. FIG. 2 is explained in conjunction with FIG. 1.

In the embodiments described herein, the mapping platform 102 may include a processing means, such as, at least one processor (hereinafter interchangeably used with processor) 202, a storage means, such as, at least one memory (hereinafter interchangeably used with memory) 204, a communication means, such as, at least one network interface (hereinafter interchangeably used with network interface) 206 and an I/O interface 208. The processor 202 may retrieve computer executable instructions that may be stored in the memory 204 for execution of the computer executable instructions. The memory 204 may store the sensor data associated with the road in the geographic region for which the map data may be generated. In accordance with an embodiment, the processor 202 may be configured to obtain input (such as, real time sensor data) from background batch data services, streaming data services or third party service providers, and renders output, such as, the generated map data, notification associated with the generated map data for use by the end user on the UE 106 through the network interface 206.

The processor 202 may be embodied in a number of different ways. For example, the processor 202 may be embodied as one or more of various hardware processing means such as a coprocessor, a microprocessor, a controller, a digital signal processor (DSP), a processing element with or without an accompanying DSP, or various other processing circuitry including integrated circuits such as, for example, an ASIC (application specific integrated circuit), an FPGA (field programmable gate array), a microcontroller unit (MCU), a hardware accelerator, a special-purpose computer chip, or the like. As such, in some embodiments, the processor 202 may include one or more processing cores configured to perform independently. A multi-core processor may enable multiprocessing within a single physical package. Additionally, or alternatively, the processor 202 may include one or more processors configured in tandem via the bus to enable independent execution of instructions, pipelining and/or multithreading. Additionally, or alternatively, the processor 202 may include one or more processors capable of processing large volumes of workloads and operations to provide support for big data analysis. In an example embodiment, the processor 202 may be in communication with the memory 204 via a bus for passing information among components of the mapping platform 102.

The memory 204 may be non-transitory and may include, for example, one or more volatile and/or non-volatile memories. In other words, for example, the memory 204 may be an electronic storage device (for example, a computer readable storage medium) comprising gates configured to store data (for example, bits) that may be retrievable by a machine (for example, a computing device like the processor 202). The memory 204 may be configured to store information, data, content, applications, instructions, or the like, for enabling the mapping platform 102 to carry out various functions in accordance with an example embodiment of the present disclosure. For example, the memory 204 may be configured to buffer input data for processing by the processor 202. As exemplarily illustrated in FIG. 2, the memory 204 may be configured to store instructions for execution by the processor 202. As such, whether configured by hardware or software methods, or by a combination thereof, the processor 202 may represent an entity (for example, physically embodied in circuitry) capable of performing operations according to an embodiment of the present disclosure while configured accordingly. Thus, for example, when the processor 202 is embodied as an ASIC, FPGA or the like, the processor 202 may be specifically configured hardware for conducting the operations described herein.

Alternatively, as another example, when the processor 202 is embodied as an executor of software instructions, the instructions may specifically configure the processor 202 to perform the algorithms and/or operations described herein when the instructions are executed. However, in some cases, the processor 202 may be a processor specific device (for example, a mobile terminal or a fixed computing device) configured to employ an embodiment of the present disclosure by further configuration of the processor 202 by instructions for performing the algorithms and/or operations described herein. The processor 202 may include, among other things, a clock, an arithmetic logic unit (ALU) and logic gates configured to support operation of the processor 202. The network environment, such as, 100 may be accessed using the network interface 206 of the mapping platform 102. The network interface 206 may provide an interface for accessing various features and data stored in the mapping platform 102.

The processor 202 of the mapping platform 102 may be configured to determine health infection conditions in the geographic region based on the sensor data. The processor 202 of the mapping platform 102 may be further configured to generate a health data record for the geographic region, based on the determined health infection conditions in the geographic region. The processor 202 of the mapping platform 102 may be further configured to generate the map data, based on determined health infection conditions in the geographic region based on the sensor data. The processor 202 of the mapping platform 102 may be further configured to map match location data associated with the geographic region to a specific location of map data for the geographic region.

The memory 204 of the mapping platform 102 may be configured to store a dataset (such as, but not limited to, the sensor data and the map data) associated with the road in the geographic region. In accordance with an embodiment, the memory 204 may include processing instructions for generating the map data with a dataset associated with the road. In accordance with an embodiment, the dataset may include real-time data and historical data, from service providers. The memory 204 of the mapping platform 102 may be configured to store the generated map data.

In some example embodiments, the I/O interface 208 may communicate with the mapping platform 102 and displays input and/or output of the mapping platform 102. As such, the I/O interface 208 may include a display and, in some embodiments, may also include a keyboard, a mouse, a joystick, a touch screen, touch areas, soft keys, one or more microphones, a plurality of speakers, or other input/output mechanisms. In one embodiment, the mapping platform 102 may comprise user interface circuitry configured to control at least some functions of one or more I/O interface elements such as a display and, in some embodiments, a plurality of speakers, a ringer, one or more microphones and/or the like. The processor 202 and/or I/O interface 208 circuitry comprising the processor 202 may be configured to control one or more functions of one or more I/O interface 208 elements through computer program instructions (for example, software and/or firmware) stored on a memory 204 accessible to the processor 202. The processor 202 may further render notification associated with the generated map data on the user equipment 106 via the I/O interface 208.

In some embodiments, the processor 202 may be configured to provide Internet-of-Things (IoT) related capabilities to users of the mapping platform 102 disclosed herein. The IoT related capabilities may in turn be used to provide smart city solutions by providing real time parking updates, big data analysis, and sensor-based data collection by using the cloud based mapping system for providing navigation and parking recommendation services using an. environment that may be accessed using the network interface 206. The network interface 206 may provide an interface for accessing various features and data stored in the mapping platform 102.

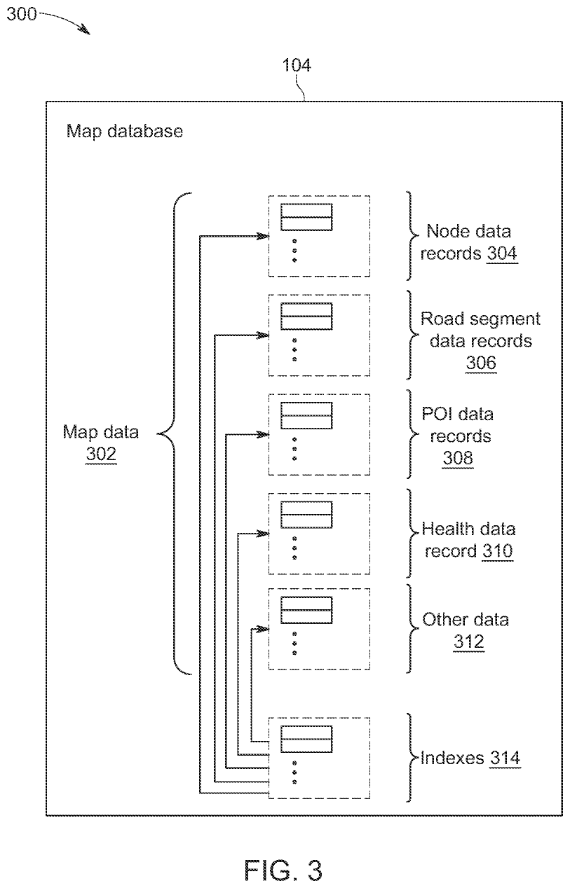

FIG. 3 illustrates a block diagram 300 of the map database 104 used to store data, such as sensor data for generation of map data, in accordance with an example embodiment. FIG.3 is explained in conjunction with FIG. 1 and FIG. 2.

With reference to FIG. 3, there are shown node data records 304, road segment or link data records 306, POI data records 308, health data record 310, other data records 312, and indexes 314 stored in the map database 104.

In one embodiment, the map database 104 may be configured to store, associate and/or link data such as, historical map data (e.g., parking data, traffic data, weather data, map feature data, health infection condition data etc.) and specialized sensor data and map data generated according to the various embodiments described herein, and/or any other information used or generated by the mapping platform 102 with respect to providing map data updates. In one embodiment, the map database 104 may include map data 302 used for (or configured to be compiled to be used for) mapping and/or navigation-related services indicative of health infection conditions, such as for route information, service information, estimated time of arrival information, location sharing information, speed sharing information, and/or geospatial information sharing, according to exemplary embodiments. The map data 302 may be collected through plurality of sources, such as sensor data from vehicles, roadside sensors, municipalities, third party sources, government agencies, health service providers and the like, and ingested into the map database 104. Further, the data collected from these pluralities of sources may then be compiled into a suitable format and stored in the map database 104. For example, the map database 104 may include node data records 304, road segment or link data records 306, POI data records 308, the health data record 310, other data records 312, and indexes 314. More, fewer or different data records can be provided.

In one embodiment, these records store map data 302 and/or features used for generating map data for the geographic region under various contexts according to the embodiments described herein. For example, the features and/or contexts include, but are not limited to: (1) functional class of the link (e.g., principal arterial roadways, minor arterial roadways, collector roadways, local roadways, etc.); (2) POI density along a link (e.g., how many POIs are located along the link); (3) night life POI density along a link (e.g., how many POIs classified related to night life are along the link, such as restaurants, bars, clubs, etc.); (4) POI types along a link (e.g., what other types of POIs are located along the link); (5) population density along a link (e.g., the population of people living or working areas around the link); (6) road density along a link (e.g., how many roads are within a threshold distance of the link); (7) zoning (e.g., CBD, residential, etc.); (8) time epoch (e.g., segmentation by a defined period of time such as 15 mins, 1 hour, etc. periods of time); (9) weekday/weekend; (10) bi-directionality (e.g., whether traffic flows in two or multiple directions along the link); and (11) accessibility to public transit (e.g., proximity to subways, buses, transit stations, etc.).

In one embodiment, the other data records 312 may include cartographic (“carto”) data records, routing data, and maneuver data. One or more portions, components, areas, layers, features, text, and/or symbols of the POI or event data can be stored in, linked to, and/or associated with one or more of these data records. For example, one or more portions of the POI, event data, or recorded route information can be matched with respective map or geographic records via position or GPS data associations (such as using known or future map matching or geo-coding techniques), for example.

In one embodiment, the indexes 314 may improve the speed of data retrieval operations in the map database 104. In one embodiment, the indexes 314 may be used to quickly locate data without having to search every row in the map database 104 every time it is accessed.

In exemplary embodiments, the road segment data records 306 are links or segments representing roads, streets, or paths, as can be used in the calculated route or recorded route information. The node data records 304 are end points corresponding to the respective links or segments of the road segment data records 306. For example, the nodes represent road intersections. The road segment data records 306 and the node data records 304 represent a road network, such as used by vehicles (like the first vehicle 114), cars, and/or other entities. Alternatively, the map database 104 may comprise path segment and node data records or other data that represent pedestrian paths or areas in addition to or instead of the vehicle road record data, for example.

The road link and nodes can be associated with attributes, such as geographic coordinates, street names, address ranges, speed limits, turn restrictions at intersections, and other navigation related attributes, as well as POIs, such as traffic controls (e.g., stoplights, stop signs, crossings, etc.), gasoline stations, hotels, restaurants, museums, stadiums, offices, automobile dealerships, auto repair shops, buildings, stores, parks, etc. The map database 104 may include data about the POIs and their respective locations in the POI data records 308. The map database 104 can also include data about places, such as cities, towns, or other communities, and other geographic features, such as bodies of water, mountain ranges, etc. Such place or feature data can be part of the POI data records 308, can be associated with POIs or POI data records 308 (such as, a data point used for displaying or representing a position of a city).

In one embodiment, the health data record 310 may include any data item associated with specialized air sensor data used by the mapping platform 102. The sensor data may include data from the specialized air sensors. In accordance with an embodiment, the sensor data may comprise location data, data associated with presence of pathogens in air for the geographic region and a combination thereof. In accordance with an embodiment, the pathogens may be associated with coronavirus family of viruses. In accordance with an embodiment, the data associated with the presence of the pathogens in air for the geographic region comprises at least one of: news related to prevalence of a disease in the geographic region. The disease may be Covid-19, medical records associated with health conditions of population in the geographic region, government records associated with presence of the health infection in the geographic region, and social network records of the population in the geographic region associated with the health infection. In accordance with an embodiment, the sensor data may comprise size data of water droplets and volume data of the water droplets. A water droplet size below a threshold range may be indicative of the health infection conditions. As such, the specialized air sensor data 310 can be associated with any of the links, map tiles, geographic areas, POIs, political boundaries, etc. represented in the map database 104.

The map database 104 can be maintained by the content provider 110A . . . 110N in association with the services platform 108 (e.g., a map developer). The map developer can collect map data 302 to generate and enhance the map database 104. There can be different ways used by the map developer to collect data. These ways can include obtaining data from other sources, such as municipalities or respective authorities. In addition, the map developer can employ field personnel to travel by vehicle along roads throughout the geographic region to observe features and/or record information about them, for example. Also, remote sensing, such as aerial or satellite photography, can be used.

The processes described herein for providing map data updates indicative of the health infection conditions may be advantageously implemented via software, hardware (e.g., general processor, Digital Signal Processing (DSP) chip, an Application Specific Integrated Circuit (ASIC), Field Programmable Gate Arrays (FPGAs), etc.), firmware or a combination thereof. Such exemplary hardware for performing the described functions is detailed below.

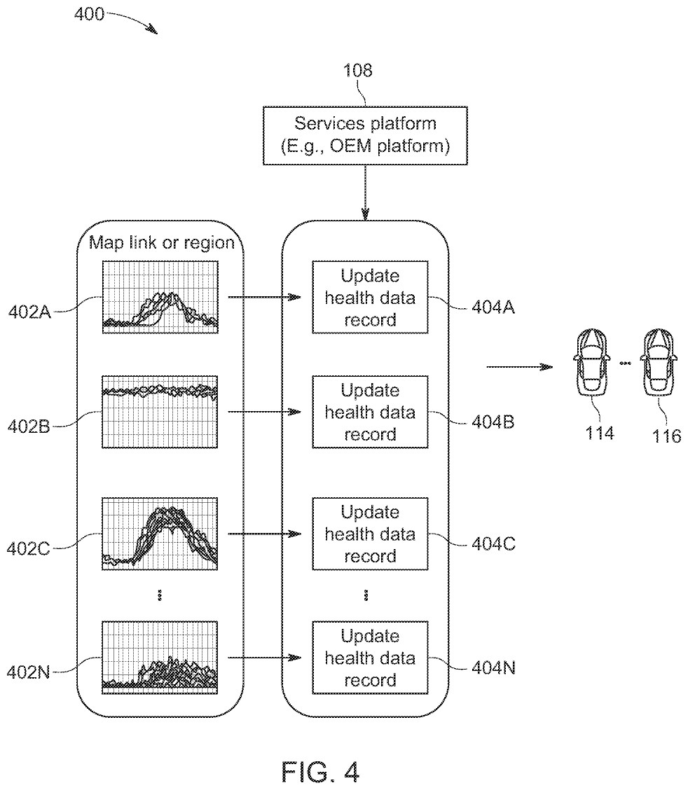

FIG. 4 is a schematic diagram that exemplarily illustrates a scenario for updating health data records of a map database indicative of health infection conditions, in accordance with an example embodiment. FIG. 4 is explained in conjunction with FIG. 1 to FIG. 3.

With reference to FIG. 4, there is shown a set of map regions 402A . . . 402N, health data records 404A . . . 404N, the services platform 108, the first vehicle 114 and the second vehicle 116.

In accordance with an embodiment, the mapping platform 102 may be configured to processes location data for the set of map regions 402A . . . 402N to generate and update health data records 404A . . . 404N (also collectively referred to as health data records 404) for transmitting updates to the first vehicle 114 and the second vehicle 116. Alternatively (e.g., when the health data records 404 have previously been determined and/or stored, for instance, in the map database 104), the mapping platform 102 can update the map data indicative of the health infection conditions by querying the map database 104 or other equivalent data store. The mapping platform 102 may be configured to provide the map data updates to the first vehicle 114 and the second vehicle 116 according to the generated map data according to the various embodiments described herein.

In accordance with an embodiment, the transmission of the generated map data that includes updated health record data is performed over a communication network (e.g., a network 112 of FIG. 1) between the mapping platform 102 and one or more user devices (e.g., the first vehicle 114, UE 106, etc.) directly. In another embodiment, the transmission is performed over the network 112 between the mapping platform 102 and a third party provider (such as, an OEM platform or other third party service. By way of example, the OEM platform or other third party platform can include the services platform 108 of FIG. 1. On receiving, the update from the mapping platform 102, the third party provider (e.g., the services platform 108 and/or any of the services 108A . . . 108N) further transmits the map data updates to one or more user devices (e.g., the first vehicle 114, UE 106, etc.).

Returning to FIG. 1, the mapping platform 102 has connectivity to map data collection infrastructure comprising, for instance, the UE 106, sensor 106B, and probe vehicles. In accordance with an embodiment, with respect to sensor data, the infrastructure can be a sensor data infrastructure comprising the specialized air sensors 106B capable of detecting number and volume of water droplets of specific size.), and then storing or utilizing the collected sensor data. In addition or alternatively, each vehicle such as the first vehicle 114 and the second vehicle 116 can be equipped with sensors (e.g., location sensors) that can also detect when the vehicle, such as the first vehicle 114 and the second vehicle 116 enters in or leaves a geographic region, for storage as health data records and may be updated as map data indicative of health infection conditions.

In one embodiment, the first vehicle 114 and/or the UE 106 associated with first vehicle 114 can act as probes traveling over a road network represented in the map database 104. For example, the UE 106 can be a standalone device (e.g., mobile phone, portable navigation device, wearable device, etc.) or installed/embedded in the first vehicle 114. In one embodiment, the first vehicle 114 and/or UE 106 may be configured with sensor 106B for determining sensor data. By way of example, the sensor 106B may include location sensors (e.g., GPS), accelerometers, compass sensors, gyroscopes, altimeters, specialized air sensor etc.

In one embodiment, when the first vehicle 114 and/or UE 106 (e.g., via a navigation system, application 106A, and/or the like) requests instructions to find parking or retrieve other map data in a given area or location for a route to travel, the mapping platform 102 can use the health data records according to the various embodiments described herein to provide updates to requested route or other map data for the requested geographic region. The mapping platform 102 can then provide the map data to the first vehicle 114 and/or UE 106 for presentation in a mapping or navigation user interface.

In one embodiment, the updated map data facilitates guiding a driver of the first vehicle 114 along a navigation route using the updated map data provided based on the updated health data records according to the various embodiments described herein. In one embodiment, as the driver navigates along the received route, the first vehicles 114 and/or UE 106 (e.g., via the application 106B) may receive real-time updates indicative of the health infection conditions on the requested map data for road links or street segments near a destination of the navigation route (e.g., links or street segments within a threshold distance of the destination).

In one embodiment, the mapping platform 102, the first vehicle 114, and/or the UE 106 can interact with the services platform 108 (e.g., an OEM platform), one or more services 108A . . . 108N, one or more content providers 110, or a combination thereof over the network 112 to provide functions and/or services based on the updated health data records. The services platform 108 (e.g., an OEM platform), one or more services 108A . . . 108N, and/or content providers 110 may provide mapping, navigation, and/or other location based services to the first vehicle 114, and/or the UE 106.

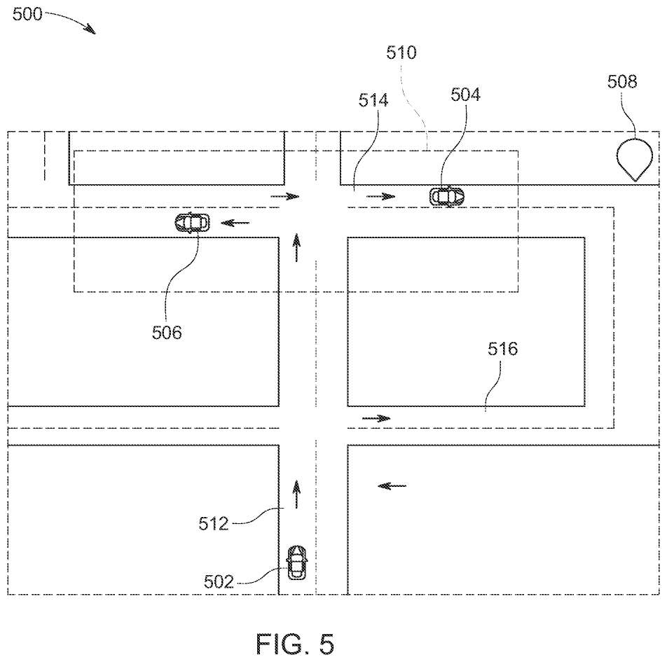

FIG. 5 is a schematic diagram 500 that exemplarily illustrates a scenario for a vehicle that uses a navigation based service indicative of health infection conditions in a geographic region, in accordance with an embodiment. FIG. 5 is explained in conjunction with FIG. 1 to FIG. 4.

With reference to FIG. 5, there is shown a vehicle 502, a first probe vehicle 504, a second probe vehicle 506, a destination location 508 for the vehicle 502, a region 510 (shown with dotted boundary) indicative of health infection conditions, and roads 512, 514 and 516.

The road 512 and the road 514 may be a part of a way leading the vehicle 502 from a source location (not shown in the FIG. 5) to the destination location 508. In one example, the road 512 may be a one-way or a two-way road. In the example embodiment illustrated in FIG. 5, the road 512 is a two-way road. Additionally, in accordance with an embodiment, the road 512 may comprise a single lane or multiple lanes, that is, the road 512 may be a single lane road, a two lane road or a four lane road.

The vehicle 502 may request for a route between two locations and the road 514 may also be a part of the requested route. The mapping platform 102 may be invoked upon receipt of the request for the route that may identify area indicative of the health infection conditions, such as the area 510 that leads the vehicle 502 towards the road 514 onto the destination location 508. In accordance with an embodiment, the route generated by the mapping platform 102, in response to the request made, may comprise a sequence of road links. Alternately, the area 510 may already be indicated in the route for the vehicle 502 and the mapping platform 102 may be invoked to generate the map data of the area 510. Irrespective of the manner in which the mapping platform 102 is triggered, the mapping platform 102 may provide measures based on the generation of the map data by the mapping platform 102 for the roads (such as, the road 514) included in the route or re-route on another road 516 towards the destination location 508. For example, the mapping platform 102 may store data indicative of presence of a health infection, such as COVID-19, in the area 510. The data may be stored in health data record 304 of mapping platform 102. When vehicle 502 traveling on road 512 is about to reach area 510, the one or more sensors installed in the vehicle 502 may trigger the mapping platform. Mapping platform 102 on being triggered, matches location of the vehicle 502 with map data stored in map database 104 of mapping platform 102, and identifies associated health data record 304. Since the area 510 in infected with the health infection, so the health record 304 indicates the presence of the health infection in area 510. The mapping platform 102 generates a response indicative of presence of health infection in area 510 and transmits the response to vehicle 502.

In some embodiments, the generated response is a route response, that indicates an alternate route of travel, such as via the road 516, for the vehicle 502.

In some embodiments, the generated response indicates a message for the vehicle 502 to switch off their air-conditioning unit while traveling through the road 512 leading to the area 510, so as to prevent infected air from reaching into the passenger compartment of the vehicle 502.

In some embodiments, the generated response indicates a message for the vehicle to switch on a heat chamber, which may be fitted in the vehicle 502 and may be specially designed to kill/destroy the disease causing pathogens that get sucked into the passenger chamber of the vehicle 502 from the outside air. The specialized heat chamber may be configured to generate extreme temperature and pressure conditions for the air sucked into the vehicle 502, to be able to destroy any accompanying disease causing pathogens.

In some embodiments, the mapping platform 102 is triggered in response to predetermined user preferences for taking an optimized route of travel, irrespective of presence of health infection in an area.

On being triggered, the mapping platform 102 may obtain the sensor data of the region encompassed by the route from a data source, such as, the map database 106B. In accordance with an embodiment, the sensor data may be obtained from the third party service providers. In accordance with an embodiment, the sensor data may comprise, for example, the data of the links that the route may encompass in the region. The sensor data of a link may comprise one or more of a link start node, a link end node, upstream heading, downstream heading, uplink data, shape location points, road classifier data, lane count or map speed limit associated with the particular link.

The mapping platform 102 may be configured to obtain data associated with presence of pathogens in air for the region. In accordance with an embodiment, such data may be obtained from specialized air sensors. The mapping platform 102 may be configured to determine health infection conditions in the region encompassed by the route based on the sensor data. The mapping platform 102 may be configured to generate the map data for the region based on the determined health infection condition in the region.

For example, the generated map data may be updated as part of a routine update, such as a monthly update. The generated map data may be used by the mapping platform 102 to predict clearance time for the health infection conditions for a particular roadway, to predict a probability of different routes, such as, shortest route, optimized route etc.; or for any other suitable purpose.

The mapping platform 102 may be further configured to obtain real time or near real time sensor data of the region encompassed by the route. The term “real-time” as used throughout the disclosure may denote that collected data that may be delivered to the mapping platform 102 in a timely manner with delays as need to transfer the data from a data collection point to the mapping platform 102. The mapping platform 102 may support real time sensor data from a huge number of vehicles for timely updates.

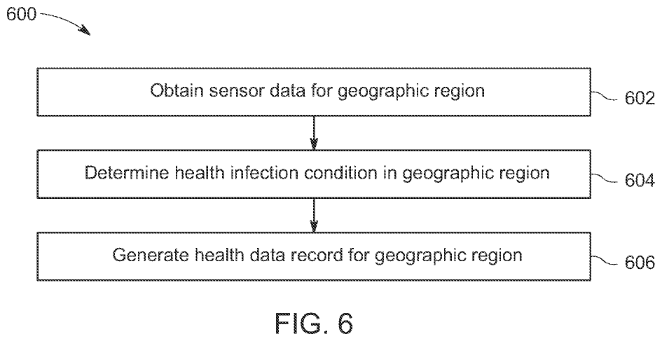

FIG. 6 illustrates a flowchart for implementation of an exemplary method to generate map data indicative of health infection conditions in a geographic region, in accordance with an example embodiment. FIG. 6 is explained in conjunction with FIG. 1 to FIG. 5.

With reference to FIG. 6, the control starts at 602. At 602, sensor data for a geographic region may be obtained. In accordance with an embodiment, the processor 202 of the mapping platform 102 may be configured to obtain sensor data for a geographic region. The sensor data may comprise location data, data associated with presence of pathogens in air for the geographic region and a combination thereof. The pathogens are associated with coronavirus family of viruses. The data associated with the presence of the pathogens in air for the geographic region comprises at least one of: news related to prevalence of a disease in the geographic region, wherein the disease is Covid-19; medical records associated with health conditions of population in the geographic region; government records associated with presence of the health infection in the geographic region; and social network records of the population in the geographic region associated with the health infection. The sensor data may also comprise size data of water droplets and volume data of the water droplets, wherein a water droplet size below a threshold range, such as 1 μm-15 μm, is indicative of the health infection conditions.

At 604, a health infection condition may be determined in the geographic region based on the sensor data. In accordance with an embodiment, the processor 202 of the mapping platform 102 may be configured to determine a health infection condition in the geographic region based on the sensor data.

At 606, a health data record may be generated for the geographic region. In accordance with an embodiment, the processor 202 of the mapping platform 102 may be configured to generate a health data record for the geographic region, based on the determined health infection conditions in the geographic region. In accordance with an embodiment, the processor 202 of the mapping platform 102 may be configured to generate the health data record for at least one of: a map tile area, a lane, a road segment, a road geometry, a shape location data record, a street, a locality, a point of interest (POI) record, a route of travel and a destination location. In accordance with an embodiment, the processor 202 of the mapping platform 102 may be configured to generate the map data for the geographic region, based on the determined health infection condition in the geographic region.

In some embodiments, when the map data has been generated to indicate the health infection conditions in the geographic region, it is used for various mapping applications. For example, location data associated with the geographic region may be map matched to a specific location of map data for the geographic region. In accordance with an embodiment, the processor 202 of the mapping platform 102 may be configured to map match location data associated with the geographic region to a specific location of map data for the geographic region.

In some embodiments, a warning message may be transmitted to at least one vehicle for indicating presence of the health infection condition on a road In accordance with an embodiment, the processor 202 of the mapping platform 102 may be configured to transmit a warning message to at least one vehicle for indicating presence of the health infection condition on a road, based on the map data.

In some embodiments, driving strategies may be recommended to the at least one vehicle, based on the generated map data.

Additionally, in some embodiments a report of the generated map data may be transmitted to a backend server.

In some other embodiments, navigation based services may be controlled for at least one vehicle based on the generated map data. The navigation services may include such as re-routing the vehicle based on the health infection conditions identified in the geographic region based on the generated map data.

In some embodiments, at least one vehicle may be controlled for selecting a vehicle functionality based on the generated map data. In accordance with an embodiment, the processor 202 of the mapping platform 102 may be configured to control operations of the at least one vehicle for selecting a vehicle functionality, based on the navigation based services. the vehicle functionality comprises switching off an air conditioning unit of the at least one vehicle, based on the navigation based services. The switching off the air conditioning unit prevents infected air from entering into a passenger compartment of the at least one vehicle. The vehicle functionality further comprises switching on a heat chamber of the at least one vehicle, based on the navigation based services. The heat chamber is configured to destroy the pathogens entering the at least one vehicle from infected air at a predefined temperature and predefined pressure.

Accordingly, blocks of the flowchart 600 support combinations of means for performing the specified functions and combinations of operations for performing the specified functions for performing the specified functions. It will also be understood that one or more blocks of the flowchart 600, and combinations of blocks in the flowchart 600, can be implemented by special purpose hardware-based computer systems which perform the specified functions, or combinations of special purpose hardware and computer instructions.

Alternatively, the mapping platform 102 may comprise means for performing each of the operations described above. In this regard, according to an example embodiment, examples of means for performing operations may comprise, for example, the processor 202 and/or a device or circuit for executing instructions or executing an algorithm for processing information as described above.

On implementing the method 600 disclosed herein, the end result generated by the mapping platform 102 is a tangible generation of map data indicative of health infection conditions for a road in a geographic region. The generation of map data indicative of health infection conditions is of utmost importance to put a halt on pandemics, for health and safety of people while they are traveling on the road in the geographic region.

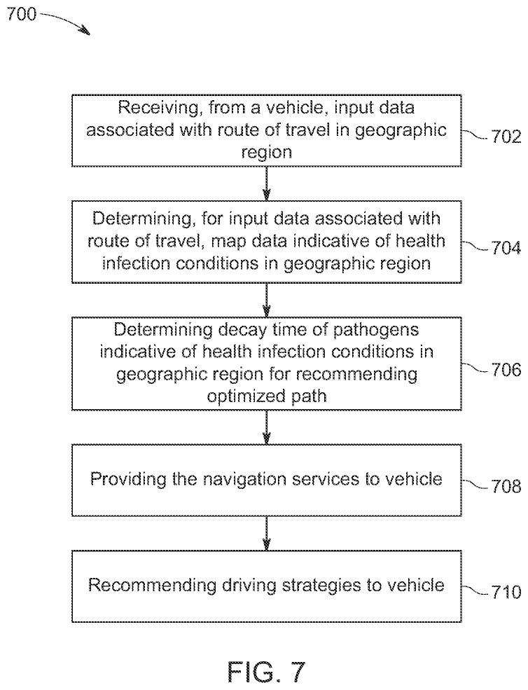

FIG. 7 illustrates a flowchart for implementation of an exemplary method for providing navigation services in a geographic region, in accordance with an example embodiment. FIG. 7 is explained in conjunction with FIG. 1 to FIG. 6.

With reference to FIG. 7, the control starts at 702. At 702, input data may be received, from at least one vehicle, associated with a route of travel in the geographic region. The processor 202 may be configured to receiving, from at least one vehicle, input data associated with a route of travel in the geographic region.

At 704, for the input data associated with the route of travel, map data indicative of health infection conditions in the geographic region may be determined. The processor 202 may be configured to determining, for the input data associated with the route of travel, map data indicative of health infection conditions in the geographic region.

At 706, decay time of pathogens indicative of the health infection conditions in the geographic region may be determined. The processor 202 may be configured to determining decay time of pathogens indicative of the health infection conditions in the geographic region for recommending optimized path. The decay time indicates time required for one of weakening of strength and reaching a state of absence of the pathogens.

At 708, navigation services may be provided to the at least one vehicle. The processor 202 may be configured to providing the navigation services to the at least one vehicle, based on the map data indicative of infected health conditions in a geographic region.

At 710, driving strategies may be recommended to the at least one vehicle. The processor 202 may be configured to recommend driving strategies to the at least one vehicle, based on the navigation services. the driving strategies comprise one or more of a rerouted path, an alternative path, a shortest path, a least infected path, a non-infected path, fastest path and an optimized path. In some embodiments, the recommendations are based on a plurality of factors, including, but not limited to, user preferences, user schedule, user social profile data, time of travel, severity of infection, presence or absence of alternate routes of travel, presence or absence of specialized heat chamber in user vehicle and the like.

Accordingly, blocks of the flowchart 700 support combinations of means for performing the specified functions and combinations of operations for performing the specified functions for performing the specified functions. It will also be understood that one or more blocks of the flowchart 700, and combinations of blocks in the flowchart 700, can be implemented by special purpose hardware-based computer systems which perform the specified functions, or combinations of special purpose hardware and computer instructions.

Alternatively, the mapping platform 102 may comprise means for performing each of the operations described above. In this regard, according to an example embodiment, examples of means for performing operations may comprise, for example, the processor 202 and/or a device or circuit for executing instructions or executing an algorithm for processing information as described above.

On implementing the method 700 disclosed herein, the end result generated by the mapping platform 102 is a tangible generation of navigation services indicative of health infection conditions for a route in a geographic region and/or destroying of pathogens in the air using the specialized heat chamber.

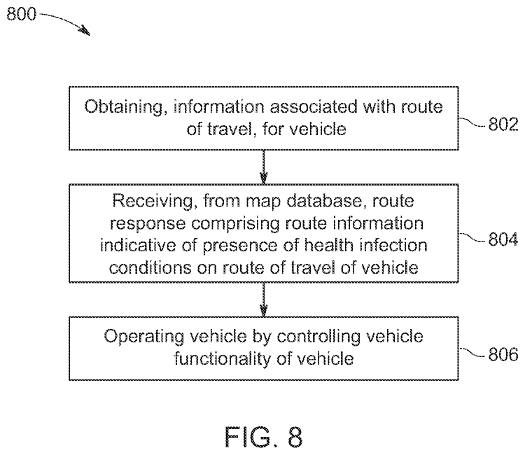

FIG. 8 illustrates a flowchart for implementation of an exemplary method for operating a vehicle, in accordance with an example embodiment. FIG. 8 is explained in conjunction with FIG. 1 to FIG. 7.

With reference to FIG. 8, the control starts at 802. At 802, information associated with a route of travel may be obtained, for the at least one vehicle. In accordance with an embodiment, a processor may be configured to obtaining, information associated with a route of travel, for the at least one vehicle.

At 804, from a map database, a route response may be received that comprises route information indicative of presence of health infection conditions on the route of travel of the at least one vehicle. In accordance with an embodiment, the processor may be configured to receiving, from a map database, a route response comprising route information indicative of presence of health infection conditions on the route of travel of the at least one vehicle; and

At 806, the at least one vehicle may be operated by controlling a vehicle functionality of the at least one vehicle. In accordance with an embodiment, the processor may be configured to operating the at least one vehicle by controlling a vehicle functionality of the at least one vehicle, based on the received route response. In accordance with an embodiment, the vehicle functionality comprises switching off an air conditioning unit of the at least one vehicle, based on the route response. In accordance with an embodiment, the vehicle functionality further comprises switching on a heat chamber of the at least one vehicle, based on the route response.

Accordingly, blocks of the flowchart 800 support combinations of means for performing the specified functions and combinations of operations for performing the specified functions for performing the specified functions. It will also be understood that one or more blocks of the flowchart 800, and combinations of blocks in the flowchart 800, can be implemented by special purpose hardware-based computer systems which perform the specified functions, or combinations of special purpose hardware and computer instructions.

On implementing the method 800 disclosed herein, the end result generated by the vehicle is a tangible generation of navigation services indicative of health infection conditions for a road in a geographic region.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

We claim:

1. A system for generating map data, the system comprising:
at least one non-transitory memory configured to store computer executable instructions; and
at least one processor configured to execute the computer executable instructions to:
obtain sensor data for a geographic region;
determine a health infection condition in the geographic region based on the sensor data; and
generate the map data for the geographic region based on the determined health infection condition in the geographic region.

2. The system of claim 1, wherein the sensor data comprises location data, data associated with presence of pathogens in air for the geographic region, or a combination thereof.

3. The system of claim 2, wherein the system further comprises one or more specialized air sensors to obtain the data associated with the presence of the pathogens in the air for the geographic region.

4. The system of claim 3, wherein the pathogens are associated with coronavirus family of viruses.

5. The system of claim 3, wherein the data associated with the presence of the pathogens in air for the geographic region comprises at least one of: news related to prevalence of a disease in the geographic region, wherein the disease is Covid-19; medical records associated with health conditions of population in the geographic region; government records associated with presence of the health infection in the geographic region; and social network records of the population in the geographic region associated with the health infection.

6. The system of claim 1, wherein the at least one processor is further configured to execute the computer executable instructions to map match location data associated with the geographic region to a specific location of map data for the geographic region.

7. The system of claim 1, wherein to generate the map data, the at least one processor is further configured to execute the computer executable instructions to generate a health data record for the geographic region, based on the determined health infection conditions in the geographic region.

8. The system of claim 8, wherein to generate the map data, the at least one processor is further configured to execute the computer executable instructions to generate the health data record for at least one of: a map tile area, a lane, a road segment, a road geometry, a shape location data record, a street, a locality, a point of interest (POI) record, a route of travel and a destination location.

9. The system of claim 1, wherein the at least one processor is further configured to execute the computer executable instructions to transmit a warning message to at least one vehicle for indicating presence of the health infection condition on a road, based on the map data.

10. The system of claim 10, wherein the at least one processor is further configured to execute the computer executable instructions to recommend driving strategies to the at least one vehicle, based on the map data.

11. The system of claim 1, wherein the at least one processor is further configured to execute the computer executable instructions to provide navigation services for at least one vehicle, based on the generated map data.

12. The system of claim 1, wherein the sensor data further comprises size data of water droplets and volume data of the water droplets, wherein a water droplet size below a threshold range is indicative of the health infection conditions.

13. A non-transitory computer readable medium having stored thereon, computer-executable instructions for causing a computer to execute operations for providing navigation services in a geographic region, the operations comprising:
receiving, from at least one vehicle, input data associated with a route of travel in the geographic region;
determining, for the input data associated with the route of travel, map data indicative of health infection conditions in the geographic region; and
providing the navigation services to the at least one vehicle, based on the map data.

14. The non-transitory computer readable medium of claim 13, wherein the operations further comprising recommending driving strategies to the at least one vehicle, based on the navigation services.

15. The non-transitory computer readable medium of claim 14, wherein the driving strategies comprise one or more of: a warning message associated with the route of travel based on the map data indicative of health infection conditions in the geographic region, a rerouted path, an alternative path, a shortest path, a least infected path, a non-infected path, fastest path and an optimized path.

16. The non-transitory computer readable medium of claim 15, wherein the operations further comprising determining decay time of pathogens indicative of the health infection conditions in the geographic region for recommending the optimized path, wherein the decay time indicates time required for one of weakening of strength and reaching a state of absence of the pathogens.

17. The non-transitory computer readable medium of claim 13, wherein the operations further comprising providing a recommendation for a vehicle functionality, wherein the vehicle functionality comprises at least one of: switching on a heat chamber of the at least one vehicle; and switching off an air conditioning unit of the at least one vehicle.

18. A method for operating at least one vehicle, the method comprising:
obtaining information associated with a route of travel for the at least one vehicle;
receiving, a route response comprising route information indicative of presence of health infection conditions on at least a portion of the route of travel of the at least one vehicle; and
operating the at least one vehicle by controlling or providing a recommendation for a vehicle functionality of the at least one vehicle, based on the received route response.

19. The method of claim 18, wherein the vehicle functionality comprises switching off an air conditioning unit of the at least one vehicle, based on the route response, wherein the switching off the air conditioning unit prevents infected air from entering into a passenger compartment of the at least one vehicle.

20. The method of claim 18, wherein the vehicle functionality further comprises switching on a heat chamber of the at least one vehicle, based on the route response, wherein the heat chamber is configured to destroy the pathogens entering the at least one vehicle from infected air at a predefined temperature and predefined pressure.
